(19) United States
(12) Patent Application Publication
(10) Pub. No.: US 2011/0022178 A1
(43) Pub. Date: Jan. 27, 2011

(54) MEDICAL DEVICES

(76) Inventors: David Malcolm Grant, Nottingham (GB); Nicholas William Botterill, Nottingham (GB)

Correspondence Address: Woodard, Emhardt, Moriarty, McNett & Henry LLP, 111 Monument Circle, Suite 3700, Indianapolis, IN, 46204-5137, US

(21) Appl. No.: 12/890,838

(22) Filed: Sep. 27, 2010

Related U.S. Application Data
(63) Continuation of application No. PCT/GB2009/000805, filed on Mar. 26, 2009.

(30) Foreign Application Priority Data
Mar. 26, 2008 (GB) 0805383.7

(51) Int. Cl. A61F 2/40; A61F 2/28; A61F 2/30; A61F 2/32; A61F 2/42; A61B 17/80; A61B 17/84

(52) U.S. Cl. 623/19.11; 623/23.6; 623/18.11; 623/22.11; 623/21.18; 623/20.14; 606/280; 606/300

(57) ABSTRACT

A medical device having body contacting surfaces provided with a film of amorphous calcium phosphate having a thickness of 1 nm to 10000 nm and an average dissolution rate calculated as the time taken to remove 95% of the film of at least 1 nm h⁻¹ as determined in accordance with ASTM Standard P1926-03. The characteristics of the film are such that the film is relatively rapidly dissolved from the surface by body fluids. This dissolution of the surface film of amorphous calcium phosphate is sufficiently fast to prevent the attachment of bacteria, thus preventing them forming a biofilm and keeping the cells in a planktonic (non-biofilm) state where they are more vulnerable to the body's defences. The invention is particularly applicable to implants (e.g. orthopaedic implants such as prosthetic hips) but may also be applied to “non-implanted” devices such as surgical instruments.

### CROSS REFERENCE TO RELATED APPLICATIONS

This application is a continuation of Application No. PCT/GB2009/000805 filed Mar. 26, 2009, which claims the benefit of GB Application No. 0805383.7 filed Mar. 26, 2009, which is hereby incorporated by reference.

The present invention relates to medical devices (e.g. implants or surgical instruments) and more particularly to such devices having surface characteristics which inhibit the attachment of bacteria to the device so as to reduce the possibility of bacterial infection in the patient arising from the device. The invention is particularly applicable (but not limited to) orthopaedic implants.

Implantable devices such as orthopaedic implants have been used successfully for many years. Orthopaedic implants may, for example, be used as a replacement for a worn or otherwise damaged body part, such as a hip joint or a knee. Further examples of orthopaedic implants are bone fixation devices (e.g. pins) to assist in the healing of fractured limbs or joints.

Orthopaedic implants frequently have a coating designed to promote a bioactive response to aid the bone tissue-implant interface. Many designs are based on coatings deposited by various physical and chemical methods (see references 1-29). These are often based on crystalline phases of calcium phosphate such as hydroxyapatite produced by plasma spraying (e.g. U.S. Pat. No. 5,763,092) where importance is given to degree of crystallinity of the coating or modifications that combine pores (e.g. U.S. Pat. No. 6,764,769) or mixtures with other crystalline phases such as those based on metal oxides (US Patent Specification 2005/0019365). These coatings all provide a crystalline component for the required chemical, physical and biological stability over the period of tissue integration which can be from months to years.

In spite of the successful use of implants over many years there is a significant risk of infection which can be a serious complication to surgery. The problem is aggravated as a result of bacterial resistance to anti-bacterial agents which would otherwise be used to control or eliminate the infection. For example, in June 2003, orthopaedic related infection had become such a concern that the Chief Medical Offer for the UK announced mandatory surveillance was to be carried out in all NHS Trusts to monitor infection rates in hip/knee joint replacement and open long bone fracture fixation surgeries. Infection rates in joint replacements are between approximately 1% and 10% depending on reporting agency and area although it is generally accepted that the incidence of infections is under-reported. Medicare data puts the rate at 3.5% and 12.8% for primary and revisions respectively. Infection associated with surgical implants is therefore a serious problem and for some operations such as Total Knee replacements (TKR) the improvements in surgical techniques are at such a level that the prime mode of failure of TKR is likely to be infection.

It is therefore an object of the present invention to provide medical devices (e.g. surgical implants) having surface characteristics which reduce the possibility of bacterial infection in the patient arising from the device.

According to a first aspect of the present invention there is provided a medical device having body contacting surfaces provided with a film of amorphous calcium phosphate having a thickness of 1 nm to 10000 nm and an average dissolution rate calculated as the time taken to remove 95% of the film of at least 1 nm h⁻¹ as determined in accordance with ASTM Standard P1926-03.

Thus, in accordance with the invention, a medical device has a thin film (1-10000 nm) coating of amorphous calcium phosphate.

By the term “amorphous” we mean that the calcium phosphate does not display any crystalline peaks in its X-Ray diffraction spectrum but rather an amorphous “hump” (see also Examples 2 and 3 infra).

The amorphous calcium phosphate film has dissolution characteristics such that it has an average dissolution rate calculated as the time taken to remove 95% by weight of the coating of at least 1 nm h⁻¹ as determined in accordance with ASTM Standard P1926-03. Measurement in accordance with this Standard will generate a plot of the total amount of calcium liberated with time and will “plateau” after a certain time depending on factors such as the nature and thickness of the initial amorphous coating. Generally, the “plateau” will represent complete removal of the amorphous calcium phosphate film. This may be checked in various ways. For example, complete removal of the film may be checked using standard techniques such as energy dispersive X-Ray (ERDX) analysis. A further possibility is that a separate measurement may be made to determine the initial amount of the amorphous calcium phosphate film on the device. The measurement may be based on a thickness determination effected using a ball crater method in which a large spherical ball erodes a crater and the crater is then measured from above using high resolution optical microscopy or, for very thin coatings, scanning electron microscopy. Using the geometry of a sphere and the diameters of the crater on the top of the thin film and the bottom of the thin film the thickness can be calculated. Thickness measurements can also be determined and/or cross checked using X-section transmission electron microscopy in which the film can be measured directly. These thickness measurements may be effected on a “reference” sample identical to, and prepared under the same conditions as, that to be subjected to the ASTM test procedure. Thus by knowing the initial total amount of amorphous calcium phosphate on the sample to be tested in accordance with the ASTM Procedure, and being able to determine therefrom the time by which 95% of the total amount has been removed from the sample, it is possible to determine the average dissolution rate for the amorphous calcium phosphate film irrespective of whether or not the “plateau” represents full removal of the coating or “simply” a point at which further coating cannot be removed.

It should be appreciated that the test for solubility of the amorphous calcium phosphate coating is not necessarily effected on the medical device per se but (possibly more conveniently) using a test sample (e.g. a titanium disk) to which an identical amorphous calcium phosphate coating has been applied.

Although for the purposes of the invention dissolution rates are defined in accordance with the ASTM Standard, measurements can be simply cross checked by using known thickness of films onto a mirror finish sample and using interference colourometric changes to visually observe the disappearance of the film or elipsometry.

The amorphous calcium phosphate has a thickness of 1 nm to 10000 nm. Thickness may be determined as described above.

The amorphous calcium phosphate film may be formed using Physical Vapour Deposition using procedures as described more fully later.

The calcium:phosphorous ratio in the amorphous calcium phosphate may, for example, be in the range of (0.1 to 2):1, more preferably (0.4 to 2):1 most preferably (1 to 2):1.

The characteristics of the amorphous calcium phosphate film on the body contacting surfaces of the device in accordance with the invention are such that the film is relatively rapidly dissolved from the surface by body fluids. This dissolution of the surface film of amorphous calcium phosphate is sufficiently fast to prevent the attachment of bacteria, thus preventing them forming a biofilm and keeping the cells in a planktonic (non-biofilm) state where they are more vulnerable to the body's defences and, in the case of an implant, any antibacterial agent applied with the implant as per standard operating procedure.

As indicated, the surface film of amorphous calcium phosphate has a dissolution rate calculated as the time taken to remove 95% of the film of at least 1 nm h⁻¹ as determined in accordance with ASTM Standard F1926-03. Preferably this rate is at least 10 nm h⁻¹ and even more preferably at least 20 nm h⁻¹. Generally the rate of dissolution will not exceed 250 nm h⁻¹. Preferably the thickness of the amorphous calcium phosphate film is 10 nm to 5000 nm, more preferably 20-1000. Generally the characteristics of the surface film of amorphous calcium phosphate will be such that the film is completely removed in a period of less than 30 h, typically 0.1-24 h, using the same conditions as employed in ASTM F1926-03. Ideally the film is completely dissolved in 1-4 h under these conditions. Complete removal of the film may be checked using standard techniques such as Energy Dispersive X-Ray (ERDX) analysis.

The invention is applicable to a wide range of devices and in particular to body implants. The invention is applicable particularly to orthopaedic and dental implants and examples include prosthetic hips, ankles, shoulders and knees as well as other orthopaedic devices such as bone plates, percutaneous devices (e.g. pins, fracture fixation devices, dental pins and cannulae). Although the invention is applicable particularly to orthopaedic implants, it may also be applied to other devices. These include non-orthopaedic implants and devices such as stents (e.g. cardiovascular or urological stents), artificial grafts, catheters, shape memory devices, surgical instruments and needles.

All devices in accordance with the invention have their body contacting surfaces provided with the thin film of amorphous calcium phosphate. By “body contacting surfaces” we mean those surfaces of the device which (when “used”, e.g. implanted) will be in contact with body tissue, which term as used herein includes but is not limited to bone. In the case, for example, of implants which in use are wholly intra-corporeal (e.g. a prosthetic hip joint) then all exterior surfaces of the prosthesis will be provided with the thin film of calcium phosphate. However the invention also extends to implants which will be partially extra-corporeal, in which case those surfaces of the implant which will be extra-corporeal need not be provided with the calcium phosphate coating.

The material forming the “structural” component of the device (e.g. implant) is not critical, subject of course to the proviso that the device has the required strength for its purpose. Thus, for example, the device may be of a metal, metal alloy or ceramic or polymeric material or composite. Examples of specific materials include, but are not limited to, titanium and its alloys, cobalt chrome alloys and stainless steel alloys, NiTi based shape memory alloys, crystalline ceramics such as hydroxyapatite, titania, alumina, zirconia, degradable and non degradable polymers such as polyglycolide, polylactide, copolymers, of glycolide and lactide, polycaprolactone and polymethylmethacrylate, polyethylene (poly(tetrafluorethylene).

The substrate onto which the amorphous calcium phosphate film is deposited may have a bioactive or optimised implant surface which (it will be appreciated) is exposed once the calcium phosphate film has been dissolved. Such a bioactive or optimised implant surface may take any form known in the art and is not critical to the present invention. Thus, for example, a substrate forming an orthopaedic implant may have a surface promoting a bioactive response to aid the bone tissue—implant interface. Examples have been given above (see also references (1-30)) and include crystalline phases of calcium phosphate such as hydroxyapatite produced by plasma spraying or modifications that combine pores or mixtures with other crystalline phases such as those based on metal oxides.

The underlying bioactive surface can be already present as on an existing commercial implant such as managed topographies, roughened, beaded or wired surfaces. In terms of chemistries these surfaces often have beneficial natural oxides such as titania or additional stable bioactive coatings such as crystalline bioceramics. These chemistries have defined crystalline structures with long term stabilities (greater than several days to years).

To explain the invention further (and whilst we do not wish to be bound by theory) it is anticipated that the following steps take place in vivo. In this case we illustrate the case of a bone contacting metal implant that has the invention of a dissoluting thin film as described above deposited on an osteoconductive surface. However the argument could be applied to other tissue types in contact with an implant.

Step 1: Implant surface with a thin film dissolution coating of Calcium Phosphate.

Step 2: During implantation bacteria colonise the top surface of the implant, along with proteins and other host cell types such as osteoblasts.

Step 3: The thin film coating goes into solution removing all the organic matter from the surface including the bacteria.

Step 4: The bacteria therefore remain in their planktonic state, that is they are unable to form a biofilm on the implant surface. This makes the bacteria vulnerable as they are not protected in their biofilm. This allows the body's defences and the anti-bacterial agents that are normally given to patients on implantation to be effective. The anti-bacterial agents are able to penetrate the bacteria and also macrophages are attracted to these free bacteria thus removing them.

Step 5 Dissolution is complete and the subsurface is revealed. In this example an osteoconductive surface is revealed which promotes protein conformation and osteoblast attachment. There is now little or no competition for this surface with bacteria or biofilms as these are now removed.

Step 6 Full osseointegration continues as osteoblasts proliferate and differentiate. The full confluence on the implant surface prevents any remaining bacteria to attach to implant resulting in a stable bacteria free tissue surrounding implant.

This may be explained in more detail as follows: Upon complete dissolution the underlying biomaterial surface or a specially deposited interlayer could provide a suitable surface for the normal cellular interaction with the surface, however as stated above by this time it is hypothesised in vivo that the bacteria will not be present. This is because during the dissolution process all of the bacteria along with proteins and other cell types are unable to attach to the surface. The bacteria therefore remain in their planktonic state, that is they are unable to form a biofilm on the implant surface. This makes the bacteria vulnerable as they are not protected in their biofilm. This allows the body's defences and the normal anti-bacterial agents that are normally given to patients on implantation to be effective. Thus when the dissolution of the coating is complete, a biocompatible or bioactive surface is revealed based on the substrate of a coated underlayer that will promote the tissue response required without bacterial colonisation.

The amorphous calcium phosphate coating may be deposited by Physical Vapour Deposition (PVD). The dissolution rate of the coating can be controlled readily by changes to the PVD conditions of power density, partial pressures of gases, chamber geometry and target geometry.

In preferred embodiments a plasma sputtering PVD methodology is used to produce a uniform amorphous calcium phosphate coating. The power supply may be RF or pulsed DC. The working gas is controlled using feedback from pressure transducers. Uniform thin film coatings are produced based on the desired dissolution time. For example for a dissolution time of 4 h and a dissolution rate of 10 nm h⁻¹ then a uniform 40 nm coating is produced. Such thin film coatings are readily produced, for example a single target can produce deposition rates of 100 nm h⁻¹. The use of multiple targets allow additional elements to added into the structure to control the dissolution behaviour.

In preferred embodiments, increased sputtering rates to increase deposition time can be achieved utilising magnetron sputtering and multiple targets.

A particular advantage of this invention is that the required thin film to be deposited can be deposited in relatively short times, i.e. less than 1 h, making it commercially attractive.

The PVD methodology can deposit this amorphous thin film coating onto any metallic or ceramic substrate and many polymeric substrates. Therefore this invention has advantages in that the coating can be applied readily to all existing metallic implants (such as those based on titanium alloys, cobalt chrome alloys and stainless steel alloys) and on top of any bioactive ceramic coatings that are applied to such implants such as crystalline hydroxyapatite or Tricalcium phosphate that are used for bone integration.

The PVD methodology with suitable jigging allows easy deposition onto 3-D objects and the thin film coating conforms with the existing surface.

The coating can be applied readily to any external shape and is ideally suited to be applied to existing orthopaedic implants providing an easy to apply and safe antimicrobial coating which does not include any pharmacological elements or anti-bacterial agents that may harm the implant's integration and reduces usage of antibiotics.

The invention will be further described by the following non-limiting examples in conjunction with the accompanying drawings, in which:

FIG. 1 demonstrates the dissolution behaviour of an amorphous calcium phosphate coating showing the results of Example 2.

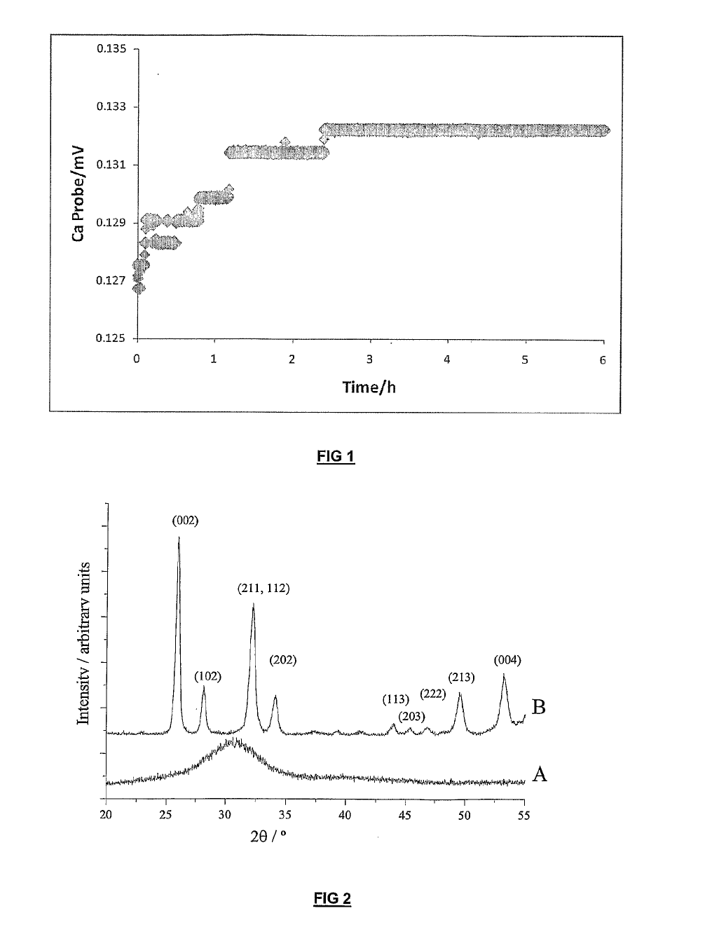

FIG. 2 shows X-Ray diffractograms illustrating the results of Example 3;

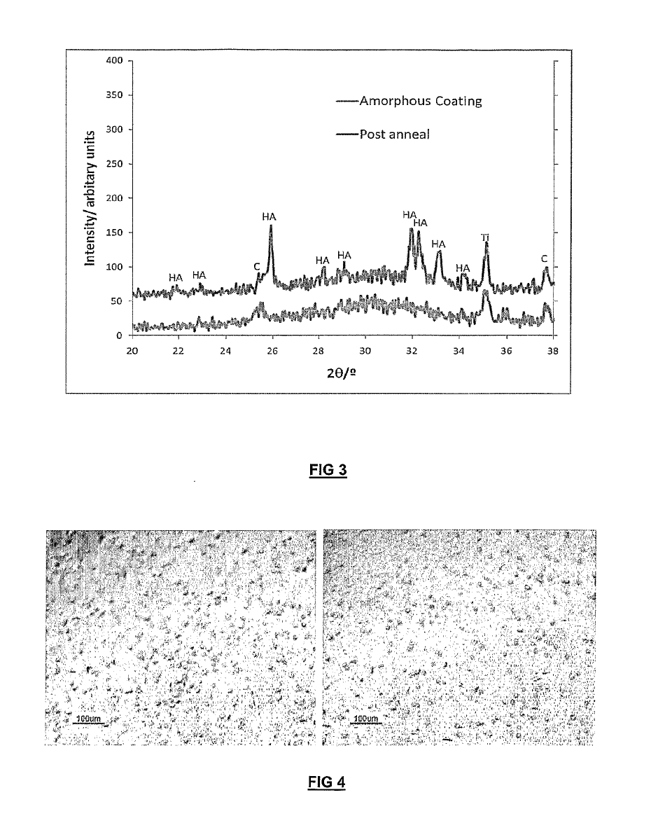

FIG. 3 shows X-Ray diffractograms illustrated the results of Example 4; and

FIGS. 4(a) and (b) are micrographs of cell cultures demonstrating the results of Example 5.

### EXAMPLE 1

This Example illustrates use of Physical Vapour Deposition to deposit thin film coatings of amorphous calcium phosphate onto 10 mm diameter disc coupons of commercial purity (CP) grade titanium.

A commercial physical vapour deposition (PVD) system (Teer Coatings UDP650) was used to generate the amorphous coatings for this work, comprising a vacuum chamber equipped with a serial pumping system (rotary and diffusion pumps). Gas pressure within the system was controlled using a feedback system comprising a capacitive manometer pressure transducer and piezo controlled mass flow control gas valve. Samples were held on a stainless steel jig, coupled to a pulsed direct current (DC) bias power supply. A 3 inch (76.2 mm) diameter Hydroxyapatite target, was mounted on a magnetron on the sidewall of the vacuum chamber (such that it was immediately adjacent to the sample holder) and connected to radio frequency (RF) power supply with an automatic capacitive/inductive tuned matching network.

Substrates were cleaned in an ultrasonic bath for 900 s each in acetone, methanol and distilled water before being dried in a Nitrogen gas stream. Samples were then introduced into the vacuum chamber, in which they were held on the stainless steel jig adjacent to the Hydroxyapatite target with a separation of circa 110 mm. The chamber was subsequently evacuated to a base pressure of circa 5×10⁻⁵ Torr.

Once base vacuum was achieved, a controlled flow of 41 sccm of Argon gas was introduced into the chamber via the aforementioned feedback system in order to raise the chamber partial pressure to circa 1×10⁻³ Torr. RF power (13.56 MHz) at a density of 3.8 Wcm⁻² was then applied to the Hydroxyapatite target, whilst simultaneously a pulsed DC bias voltage of −25 V (frequency 250 kHz pulse width 500 ns) was applied to the sample holder, thereby striking a plasma within the process chamber. Such conditions were maintained for a period of 1800 s before DC and RF power were switched off and samples allowed to cool for a period of 900 s before the chamber was vented to atmosphere to facilitate sample removal.

The thickness of the deposited film was measured by ball crater measurements and cross-section Transmission Electron Microscopy (TEM). For a given set of conditions (power density and type, gas composition, partial pressure, target and sample distance from target and sample bias), it was found that the thickness of film deposited was linear with time. Samples of thickness 25, 50, 80, 100 and 200 nm were used to test the linearity for a similar sample distance, in this case 110 mm, from the target which showed a deposition rate of ca. 100 nm h⁻¹.

### EXAMPLE 2

Using the procedure of Example 1, a coating of amorphous calcium phosphate having a thickness of 50 nm was deposited onto a titanium disk coupon.

The dissolution behaviour of the amorphous calcium phosphate coating with time was then determined in accordance with ASTM Standard F1926 using a calcium probe.

The result is shown in FIG. 1 which is a plot of, in effect, amount of calcium dissolved from the coating relative to time as measured using a calcium probe. It will be noted that the maximum amount of calcium was dissolved from the coating after about 2.5 h, as represented by the beginning of the upper plateau.

Scanning electron microscopy on the surface and Energy Dispersive X-Ray analysis confirmed no elements of calcium or phosphorus were present on the surface to a sensitivity of 1%.

Therefore from the plot of FIG. 1 it can be seen that the average dissolution rate of the coating was ca 20 nm h⁻¹.

### EXAMPLE 3

Using the procedure of Example 1, a coating of amorphous calcium phosphate having a thickness of 200 nm was deposited on to a silicon wafer and examined by glancing angle X-Ray diffraction (using Cu Kα X-Rays). The resulting X-Ray diffractogram (a plot of Intensity (arbitrary units) vs 2θ) is shown as curve (A) in FIG. 2. Despite using glancing angle X-Ray diffraction the X-Rays will penetrate into the substrate thus the advantage of using a silicon wafer is that no substrate peaks are in the region of 2θ of interest. It will be noted that, curve A displays the classic signature of an amorphous material with no observed crystalline peaks and an amorphous “hump” distributed around 30° in 2θ.

To demonstrate the purity of the deposited coating, the latter was recrystallised by annealing at 600° C. for 2 h in flowing argon. The diffractogram of the annealed coating is shown as curve (B) in FIG. 2 and clearly demonstrates a crystalline diffraction pattern for hydroxyapatite.

### EXAMPLE 4

Using the procedure of Example 1, an amorphous coating having a thickness of 800 nm was deposited onto a titanium disc coupon. In this case, the thicker coating of 800 nm (as compared to 200 nm in Example 2) was used to improve the signal compared to the X-Ray diffraction from the titanium substrate. The X-Ray diffractogram for the amorphous coating is shown as the lower curve in FIG. 3 and again confirms the amorphous nature displaying the classic signature of an amorphous material with no observed crystalline peaks and an amorphous “hump” distributed around 30° in 2θ. The crystalline peaks that are observed in the diffractogram for the amorphous coating come from the pure titanium substrate (Ti) and corundum (C) sample holder.

The amorphous coating was then subjected to recrystallisation using a post-anneal up to 700° C. in He. The X-Ray diffractogram of the post-annealed (recrystallised) material is shown in the upper curve of FIG. 3 from which a reduction in the amorphous hump is observed together with the emergence of crystalline peaks of hydroxyapatite confirming the phase purity of the coating. The other diffraction peaks are from the Ti substrate and corundum (C) holder.

### EXAMPLE 5

Using the procedure of Example 1, samples were produced having a 50 nm thick coating of amorphous calcium phosphate on the titanium coupons.

Using one sample, the dissolution rate of the coating (calculated by the time to remove 95% of the coating) was tested in accordance with ASTM Standard F1926-03 and found to be 20 nm h⁻¹.

A further sample of the titanium coupon with the 50 nm thick coating of amorphous calcium phosphate was used to test the ability for biological cells to attach and spread in vitro. For this purpose, fibroblast cells were seeded at a cell density of 40,000 cells cm⁻² on to the surface of the amorphous calcium phosphate coating (deposited on the titanium coupon) and also onto an untreated titanium coupon.

FIGS. 4 (a) and (b) show the results 111 min after initial seeding on the titanium and amorphous calcium phosphate surface respectively. As shown in FIG. 4(b) the cells remained rounded and unwilling to spread on the amorphous calcium phosphate surface. In contrast, the cells on the titanium (FIG. 4(a)) showed signs of adhesion and spreading.

### EXAMPLE 6

An amorphous dissolution layer typically 200 nm thick based on calcium phosphate was deposited by the Physical Vapour Deposition method as outlined in Example 1 onto 10 mm discs of CP grade Ti. Half of these discs were then recrystallised at 600° C. under flowing Argon for 2 h to produce a stable crystalline hydroxyapatite thin film coating to be used to compare with the amorphous dissolution coating. Onto these two types of samples human osteoblasts were seeded and measurements of attachment made at a time point of 90 minutes. Blank wells of tissue culture plastic were used as controls. Detail of the culturing method and attachment studies is as follows:

Human Osteoblast Cells (HOBS) were cultured to confluence on tissue culture plastic (TCP) in 500 ml Dulbecco/Vogt Modified Eagle's Minimal Essential Medium (DMEM) supplemented with 10% fetal bovine serum, 1% L-Glutamine, 2% HEPES Buffer, 1% non-essential amino acids, 2% penicillin and streptomycin (Invitrogen, UK) and 75 mg ascorbic acid (Sigma, UK) and maintained at 37° C. and 5% CO₂. Once the cells were confluent then the cells were washed in phosphate buffer saline (PBS) and trypsanised, centrifuged and resuspended. Cell number was assessed using a trypan blue exclusion stain which stains non-viable cells. An aliquot of 50 μl of trypan blue was mixed with 50 μl of cell suspension, applied to a haemocytometer and placed under a Nikon Eclipse TS100 microscope. A viable cell count was the calculated for the total number of cells.

For cell attachment studies the coated disc samples were placed into a 24 well plate (Nunc, UK) with one sample per well and 1 ml of media was added to each well, blank wells of tissue culture plastic were used as the control. Tests were run in triplicate and then repeated. Cells were seeded into wells at a cell density of 10,000 cells cm⁻². Plates were incubated for 90 minutes at 37° C. and 5% CO₂. After this period, media was removed and the samples were washed in PBS three times and fixed in 4% paraformaldehyde for 10 min. Samples were washed again and then permealised for 5 min at −20° C. and washed. 0.1% Propidium Iodide was added for 1-2 s before washing and mounting samples on glass slides. A PBS solution containing 2 mg/ml of 1,4-diazabicyclo[2.2.2]octane (DABCO) was prepared at pH 8.6 and combined with glycerol in a 9:1 ratio of glycerol:DABCO/PBS. This solution and a coverslip were then applied for imaging. A Lecia DMLB optical microscope with a mercury lamp used in fluorescence mode with a BG38 filter at ×10 magnification with a Nikon DXM1200 digital camera was used to image 25 random visual fields per sample. Cell counting was performed manually.

The results of this study showed that the stable crystalline Hydroxyapatite coating had a similar number of HOBs attached as the control. This was quantified as number of HOBs attached as a percentage of the control as 90±3%. However, there was a clear visual reduction of the number of HOBs that were attached to the amorphous calcium phosphate coating and this was quantified as 50±20% of the control showing the effectiveness of the dissoluting layer in reducing the number of attached cells after 90 min.

### EXAMPLE 7

In this in vitro experiment, bacterial attachment was assessed on a 80 nm coating of amorphous calcium phosphate (produced as in Example 1) on a commercially produced medical grade Ti6Al4V titanium alloy coupon of 12 mm diameter with a grit blast finish (typical of hip stem implants). The calcium phosphate layer was one capable of dissolving in 4 hours and had a dissolution rate of 20 nm h⁻¹ as determined by methods detailed above.

Bacterial methodology was as follows. 100 μl of 10⁷ cfu/ml S. aureus suspension was applied to the samples and allowed to dry on at 37° C. Samples were then transferred to a flow cell and fresh media flowed over the samples for 4 hours at 37° C. Samples were removed and stained using BacLight Live/Dead stain. Samples were imaged (blinded) on a laser scanning confocal microscope using a 64× oil immersion objective, in which 3 random images were taken from each sample. Images were scored from 0 to 3, according to the number of colonies which could be counted in each field: 0=none/1-3 colonies; 1=4-10 colonies; 2=10-20 colonies; 3=20+colonies/communities/biofilm. Scores were totalled and averaged for the 3 images from each sample replicate. The average of the 6 replicates for each sample type was then taken.

The results of the experiment and the repeat experiment showed an average score based on 72 images from 24 samples without the dissoluting layer to be of 2.5±0.12 while with a dissoluting layer the average score was only 0.56±0.14. A clear indication of the effectiveness of the thin film dissoluting layer in reducing bacteria colonisation.

Example 7 thus shows that the same difficulty of attachment as shown in Examples 5 and 6 for different cell types also take place for bacteria thus preventing and restricting bacteria adhesion and preventing or reducing biofilm formation.

### REFERENCES

1. K. de groot, R. Gessink, C. P. A. T, Klien and P. Serekian, Plasma sprayed coatings of hydroxyapatite, Journal of biomedical materials research, 21 (1987) 1375-1381.
2. L. Chou, B. Marek, W. R. Wagner, Effects of hydroxyapatite coating crystallinity on biosolubility, call attachment efficiency and proliferation in vitro, Biomaterials, 20 (1999) 977-985.
3. C. Y. Yang, B. C. Wang, T. M. Lee, E. Chang and G. L. Chang, Intradmedulluary implant of plasma-sprayed hydroxyapatite coating: An interface study, Journal of biomedical materials research, 36 (1997)39-48.
4. J. L. Ong, D. L. Carnes, K. Bessho, Evaluation of titanium plasma-sprayed and plasma-sprayed hydroxyapatite implants in vivo, Biomaterials, 25 (2004) 4601-4606.
5. K. Anselme, P. Sharrock, P. HArdouin and M. Dard, Journal of biomedical materials research, 34 (1997) 247-259.
6. P. Frayssinet, F. Tourenne, N. Rouquet, P. Conte, C. Delga and G. Bonel, Comparative biological properties of HA plasma-sprayed coatings having different crystallinities, Journal of materials science:materials in medicine, 5 (1994) 11-17.
7 P. A. Ramires, A. Wennerberg, C. B. Johansson, F. Cosentino, S. Tundo and E. Milella, Biological behaviour of sol-gel coated dental implants, Journal of materials science:materials in medicine, 14 (2003) 539-545.
8. H-.W. Kim, H—. E. Kim and J. C. Knowles, Flour-hydroxyapatite sol-gel coating on titanium substrate for hard tissue implants, Biomaterials, 25 (2004) 3351-3358.
9 L. Gan and R. Pilliar, Calcium phosphate sol-gel-derived thin films on pourous-surfaced implants for enhanced osteoconductivity, Biomaterials, 25 (2004) 5303-5312.
10. L-. S. Kim and P. N. Numta, Sol-gel synthesis and characterization of nanostructured hydroxyapatite powder, Materials science and engineering B, 111 (2004) 232-236.
11. A. Bigi, E. Boanini and K. Rubini, Hydroxyapatite gels and nanocrystals prepared through a sol-gel process, Journal of solid state chemistry, 177 (2004) 3092-3098.
12. S. Deb, J. Giri, S. Dasgupta, D. Datta and D. Bahadur, Synthesis and characterization of biocompatible hydroxyapatite coated ferrite, Bullitin of materials science, 26 (2003) 655-660.
13. Y. Yang, K-. H. Kim and J. L. Ong, A review on calcium phosphate coatings produced using a sputtering process-an alternative to plasma spraying, Biomaterials 26 (2005) 327-337.
14. J. L. Ong, L. A. Harris, L. C. Lucas, W. R. Lacefield and D Rigney, X-Ray photoelectron spectroscopy characterization of ion-beam sputter-deposited calcium phosphate coatings, Journal of the American ceramic society, 74 (1991) 2301-2304.
15. E. S. Thian, J. Huang, S. M. Best, Z. H. Barber and W. Bonfield, Magnetron co-sputtered silicon-containing hydroxyapatite thin films-an in vitro study, Biomaterials, in press.
16. M. Hamdi and A. lde-Ektessabi, Preparation of hydroxyapatite by ion beam assisted simultaneous vapour deposition, Surface & coatings technology, 163-164 (2003) 362-367.
17. G. C. Allen, E. Ciliberto, I. Fragala and G. Spoto, Surface and bulk study of calcium phosphate bioceramics obtained by metal organic chemical vapour deposition, Nuclear instruments and methods in physics research C 116 (1996) 457-460.
18. E. N. Antonov, V. N. Bagratashvili, V. K. Popov, E. N. Sobol, M. C. Davies, S. J. B. Tendler, C. J. Roberts and S. M. Howdle, Atomic force microscopic study of the surface morphology of apatite films deposited by pulsed laser ablation, Biomaterials, 18 (1997) 1043-1049.
19. E. Jimenez, J. L. Arias, B. Leon, M. Perez-Amor, Electric discharge assisted pulsed laser deposition of hydroxylapatite, Thin Solid Films, 453-454 (2004) 422-426.
20. M. D. Ball, S. Downes, C. A. Scotchford, E. N. Anotonov, V. N. Bagratashvili, V. K. Popov, W. J. Lo, D. M. Grant and S. M, Howdle, Biomaterials, 22 (2001) 337-347.
21 P. Baeri, L. Torrisi, N. Marino and G. Foti, Ablation of hydroxyapatite by pulsed laser irradiation, Applied Surface Science, 54 (1992) 210-214.
22. A. Bigi, B. Bracci, F. Cuisinier, R. Elkaim, M. Fini, I. Mayer, I. N. Mihailescu, G. Socol, L. Struba and P. Torricelli, Biomaterials, in press.
23. M. Katto, M. Nakamura, T. Tanaka, T. Matsutani, M. Kuwata and T. Nakayama, Surface & coatings technology, 169-170 (2003) 712-715.
24. L. Cleries, J. M. Fernandez-Pradas, G. Sardin and J. L. Morenza, Biomaterials, 19 (1998) 1483-1487.
25. L. C. Nistor, C. Ghica, V. S. Teodorescu, S. V. Nistor, M. Dinescu, D. Matei, N. Frangis, N. Vouroutzis and C. Liutas, Materials Research Bullitin, 39 (2004) 2089-2101.
26. S. Kannan, A. Balamurugan and S. Rajeswari, Development of calcium phosphate coatings on type 316L SS and their in vitro response, Trends in Biomaterial and artificial organs, 16 (2002) 8-11.
27 P. Ducheyne, S. Radin, M Heughbaert and J. C. Heughebaert, Calcuim phosphate ceramic coatings on porous titanium:effect of structure and composition on electrophoretic deposition, vacuum sintering and in vitro dissolution, Biomaterials, 11 (1990) 244-254.
28.1. Zhitomirsky, Electrophoretic and Electrolytic deposition of ceramic coatings on carbon fibers, Journal of the European ceramic society, 18 (1998) 849-856.
29. C. Wang, J. Ma, W. Cheng and Ruifang Zhang, Thick hydroxyapatite coatings by electrophoretic deposition, Materials Letters, 57 (2002) 99-105.

1. A medical device having body contacting surfaces provided with a film of amorphous calcium phosphate having a thickness of 1 nm to 10000 nm and an average dissolution rate calculated as the time taken to remove 95% of the film of at least 1 nm h⁻¹ as determined in accordance with ASTM Standard P1926-03.

2. A device as claimed in claim 1 wherein said dissolution rate is at least 10 nm h⁻¹.

3. A device as claimed in claim 2 wherein said dissolution rate is at least 20 nm h⁻¹.

4. A device as claimed in claim 1 wherein the said dissolution rate does not exceed 250 nm h⁻¹.

5. A device as claimed in claim 1 wherein said film has a thickness of 10 nm to 5000 nm.

6. A device as claimed in claim 5 wherein said film has a thickness of 20 nm to 1000 nm.

7. A device as claimed in claim 1 wherein the film is completely removed in a period of less than 30 h using the same dissolution conditions as employed in ASTM F1926-03.

8. A device as claimed in claim 7 wherein the film is completely removed in a period of 0.1-24 h under said conditions.

9. A device as claimed in claim 8 wherein the film is completely removed in a period of 1-4 h under said conditions.

10. A device as claimed in claim 1 which is an implantable device.

11. A device as claimed in claim 10 which is an orthopaedic implant.

12. A device as claimed in claim 11 which is a prosthetic hip, ankle, shoulder or knee, a bone plate, pin, or fracture fixation device.
